(19) United States
(12) Patent Application Publication
(10) Pub. No.: US 2023/0323589 A1
(43) Pub. Date: Oct. 12, 2023

(54) FAST SANITIZATION SYSTEM IN A DRYER APPLIANCE

(71) Applicant: Haier US Appliance Solutions, Inc., Wilmington, DE (US)

(72) Inventors: Alexander B. Leibman, Prospect, KY (US); Kenneth G. Rudolph, Louisville, KY (US); Nemetalla Salameh, Louisville, KY (US)

(21) Appl. No.: 17/716,105

(22) Filed: Apr. 8, 2022

(51) Int. Cl. D06F 58/20; A61L 2/18

(52) U.S. Cl. CPC D06F 58/203; A61L 2/18

(57) ABSTRACT

A dryer appliance for quickly sanitizing articles of laundry includes a cabinet containing a rotatable drum defining a chamber. A drawer on the front side of the cabinet may slide between a closed position within the cabinet and an open position extended from the front side of the cabinet. The drawer may include one or more compartments for storing treatment materials, such as sanitizing agents. One or more pumps may be in fluid communication with the one or more compartments and a fluid distribution element that extends through the drum. One or more valves may be disposed between the one or more compartments and the one or more pumps to control the flow of treatment materials. Activation of the pump and opening of the one or more valves causes treatment fluid to be discharged through the fluid distribution element and into the chamber, treating articles of laundry therein. Water from a domestic water supply may also be introduced to the one or more pumps to dilute the treatment materials being used. Sanitizing treatment materials may thus be applied to articles of laundry using a variety of treatment techniques, including heated and unheated cycles, tumbling and non-tumbling cycles, cycles with or without operation of an air circulation fan, and various combinations thereof.

### FIELD OF THE INVENTION

The present subject matter relates generally to dryer appliances, or more specifically, to systems for quickly sanitizing laundry in a dryer appliance using sanitizing fluids.

### BACKGROUND OF THE INVENTION

With the onset of COVID-19, consumer awareness of and demand for the ability to sanitize clothing has dramatically risen in recent years. Various sanitization treatments have become available, requiring manual application of the sanitizing treatment to saturate each individual article of clothing prior to or subsequent to washing or drying the clothing. This process is labor intensive, requiring a substantial amount of time, large quantities of the sanitizing agent, and necessarily resulting in inhalation and residue from the sprayed sanitizing agent.

Conventional dryer appliances generally include a cabinet having a rotating drum with a chamber therein to permit drying of articles of clothing and other laundry. Some conventional dryer appliances also include sanitization cycles that do not employ any sanitizing agent. Rather, such conventional dryer appliances employ heat or a combination of heat and steam to sanitize clothing loads. In either case, heat is the primary agent for sanitizing the clothing. To be effective, the dryer appliance must heat the dryer chamber to about 160 degrees Fahrenheit and maintain that temperature for extended period of time (about 90 minutes generally). While this provides adequate sanitization, the energy and time consumed by the process is undesirable. Furthermore, some items that one may wish to launder cannot be exposed to the heat or steam required for the sanitization process. For other items, such as shoes for example, it may be undesirable to introduce them into the tumbling typically used in such sanitization cycles.

Accordingly, a dryer appliance capable of more quickly sanitizing laundry, and thus saving time and limiting energy consumption of the dryer appliance, is desirable. Further, a laundry appliance that can operate with or without heat or tumbling of the drum is desirable. In particular, a laundry appliance for automatically and efficiently applying sanitizing fluids during a drying cycle is needed. It is further desirable that such an appliance would prevent spraying of said sanitizing fluid in open spaces where it could be inhaled by users or otherwise create residue.

### BRIEF DESCRIPTION OF THE INVENTION

Advantages of the invention will be set forth in part in the following description, or may be apparent from the description, or may be learned through practice of the invention.

In one exemplary embodiment, a dryer appliance is provided, including a cabinet having a front side, a rotatable drum within the cabinet defining a chamber for the receipt of articles for drying, a drawer on the front side of the cabinet, a pump, and a fluid distribution element for dispensing treatment materials into the chamber. The drawer may be slideable between a closed position and an open position and may include a compartment for holding treatment materials. The pump may be in fluid communication with the compartment of the drawer. The fluid distribution element may be in fluid communication with the pump.

In another exemplary embodiment, a dryer appliance is provided. The appliance includes a cabinet having a front side, a rotatable drum within the cabinet defining a chamber for the receipt of articles for drying, a fan for circulating air through the chamber; a drawer on the front side of the cabinet, one or more pumps, a fluid distribution element for dispensing treatment materials into the chamber, a first valve, a second valve, and a controller. The drawer may be slideable between a closed position and an open position and may include a first compartment and a second compartment for holding treatment materials. The one or more pumps may be in fluid communication with the first compartment and the second compartment of the drawer. The fluid distribution element may be in fluid communication with the one or more pumps. The first valve may be interposed between the first compartment and one of the one or more pumps for controlling the flow of treatment materials. The second valve may be interposed between the second compartment and one of the one or more pumps for controlling the flow of treatment materials. The controller may be in communication with the fan, the one or more pumps, the first valve, and the second valve. The controller may be configured, during a dryer cycle, to deactivate the fan, activate the pump, open the first valve after deactivating the fan, close the first valve, and open the second valve after closing the first valve.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures.

FIG. 1 provides a perspective view of a dryer appliance with a closed drawer according to an exemplary embodiment of the present disclosure with portion of a cabinet of the exemplary dryer appliance removed or transparent to reveal certain components of the exemplary dryer appliance.

FIG. 2 provides a perspective view of a dryer appliance with an open drawer according to an exemplary embodiment of the present disclosure with portion of a cabinet of the exemplary dryer appliance removed or transparent to reveal certain components of the exemplary dryer appliance.

FIG. 3 provides a perspective view of an exemplary drawer according to an exemplary embodiment of the present disclosure.

FIG. 4 provides a top view a dryer appliance with a closed drawer according to an exemplary embodiment of the present disclosure with portion of a cabinet of the exemplary dryer appliance removed or transparent to reveal certain components of the exemplary dryer appliance.

FIG. 5 provides a top view of a pump, inlet conduits, a water conduit, an outlet conduit, and fluid control valves according to an exemplary embodiment of the present disclosure.

FIG. 6 provides a sides view of a pump, inlet conduits, a water conduit, and an outlet conduit, according to an exemplary embodiment of the present disclosure.

FIG. 7 provides a front perspective view of portions of an exemplary fluid distribution element in an exemplary dryer appliance with portion of a cabinet of the exemplary dryer appliance removed or transparent to reveal certain components of the exemplary dryer appliance.

Repeat use of reference characters in the present specification and drawings is intended to represent the same or analogous features or elements of the present invention.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

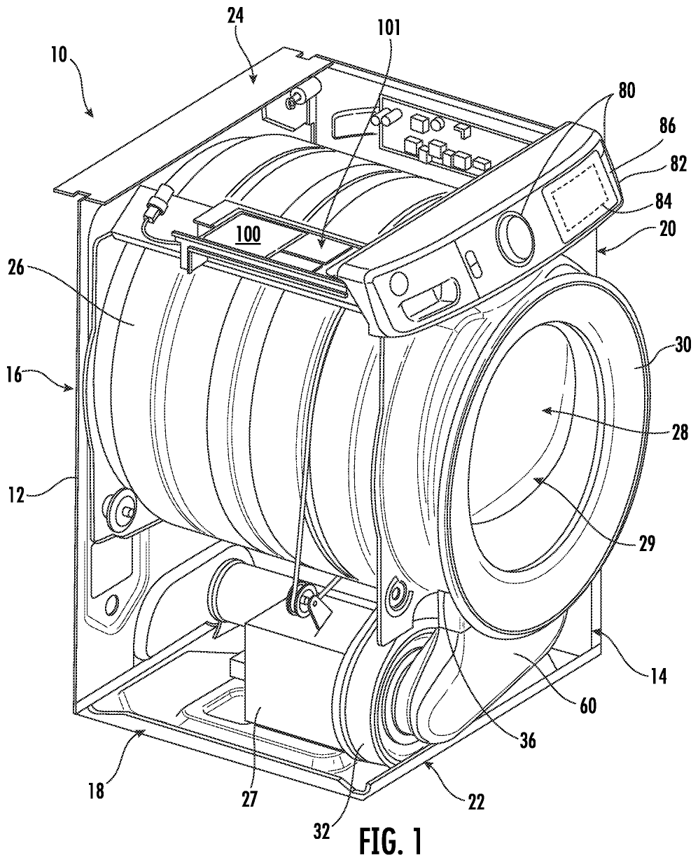

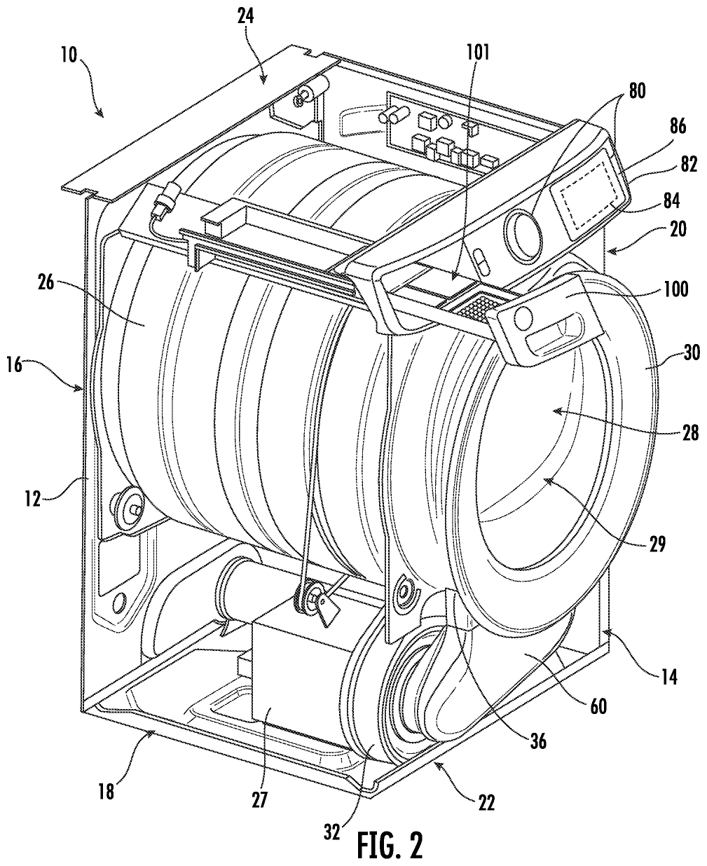

FIGS. 1 and 2 illustrate a dryer appliance 10 according to an exemplary embodiment of the present subject matter. Specifically, a portion of a housing or cabinet 12 of dryer appliance 10 is shown removed in FIGS. 1 and 2 in order to show certain components of dryer appliance 10. While described in the context of a specific embodiment of a dryer appliance, using the teachings disclosed herein it will be understood that dryer appliance 10 is provided by way of example only. Other appliances, including front or top-loaded washer appliances, or combination washer/dryer appliances, may also be utilized with the present subject matter as well.

Dryer appliance 10 defines a vertical direction V, a lateral direction L, and a transverse direction T. The vertical direction V, lateral direction L, and transverse direction T are mutually perpendicular and form an orthogonal direction system. Cabinet 12 includes a front panel 14 and a rear panel 16 spaced apart along the transverse direction T, a first side panel 18 and a second side panel 20 spaced apart along the lateral direction L, and a bottom panel 22 and a top surface or cover 24 spaced apart along the vertical direction V. Within cabinet 12 is a container or drum 26 which defines a chamber 28. Chamber 28 defines a front opening 29 for receipt of articles, e.g., clothing, linen, etc., for drying. Drum 26 extends between a front portion and a back portion, e.g., along the transverse direction T. In example embodiments, drum 26 is rotatable, e.g., about an axis that is parallel to the transverse direction T, within cabinet 12. Rotation of drum 26 is driven by drum motor 27. A door 30 is rotatably mounted to cabinet 12 for providing selective access to drum 26. Door 30 rotates between an open position and a closed position. When in the closed position, door 30 covers front opening 29.

As best shown in FIGS. 1 and 2, a fan 32 is configured for drawing air through chamber 28 of drum 26, e.g., in order to dry articles located therein, as discussed in greater detail below. Specifically, fan 32 may be in mechanical communication with motor 27, such that motor 27 rotates fan 32. In alternative example embodiments, dryer appliance 10 may include an additional motor (not shown) for rotating fan 32 independently of drum 26.

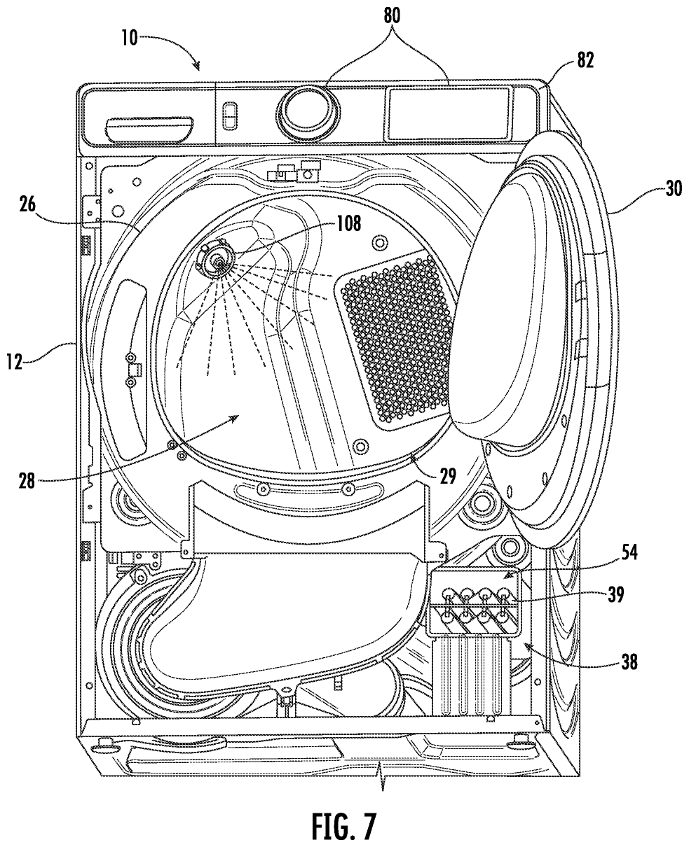

Drum 26 may be configured to receive heated air that has been heated by a heating assembly 38 (FIG. 7) in order to dry damp articles disposed within chamber 28 of drum 26. The heating assembly includes a heater 39 that is in thermal communication with chamber 28. For instance, heater 39 may include one or more electrical resistance heating elements or gas burners, for heating air being flowed to chamber 28. As discussed above, during operation of dryer appliance 10, motor 27 rotates fan 32 such that air is drawn through chamber 28 of drum 26. In particular, ambient air enters an air entrance passage defined by heating assembly due to fan 32 urging such ambient air into entrance 54. Such ambient air is heated within the heating assembly and exits as heated air. Fan 32 draws such heated air to drum 26.

Within chamber 28, the heated air can remove moisture, e.g., from damp articles disposed within chamber 28. This internal air flows in turn from chamber 28 through an outlet assembly positioned within cabinet 12. The outlet assembly generally defines an air exhaust passage 36 and includes a trap duct 60, fan 32, and an exhaust conduit (not pictured) in fluid communication with trap duct 60 via fan 32. During a drying cycle, internal air flows from chamber 28 through trap duct 60 to fan 32, e.g., as an outlet flow portion of airflow. Air further flows through fan 32 and to the exhaust conduit.

The internal air is exhausted from dryer appliance 10 via the exhaust conduit. In some embodiments, an external duct is provided in fluid communication with the exhaust conduit. For instance, the external duct may be attached (e.g., directly or indirectly attached) to cabinet 12 at rear panel 16. Any suitable connector (e.g., collar, clamp, etc.) may join the external duct to the exhaust conduit. In residential environments, the external duct may be in fluid communication with an outdoor environment (e.g., outside of a home or building in which dryer appliance 10 is installed). During a dry cycle, internal air may thus flow from the exhaust conduit and through the external duct before being exhausted to the outdoor environment.

In exemplary embodiments, trap duct 60 may include a filter portion which includes a screen filter or other suitable device for removing lint and other particulates as internal air is drawn out of chamber 28. The internal air is drawn through the filter portion by fan 32 before being passed through the exhaust conduit. After the articles have been dried (or a drying cycle is otherwise completed), the articles are removed from drum 26, e.g., by accessing chamber 28 by opening door 30. The filter portion may further be removable such that a user may collect and dispose of collected lint between drying cycles.

One or more selector inputs 80, such as knobs, buttons, touchscreen interfaces, etc., may be provided on a front control panel 82 (or on a rear control panel on a top-loading dryer, in alternative embodiments) and may be in communication with a processing device or controller 84. Signals generated in controller 84 operate motor 27 and other system components in response to the position of selector inputs 80. Additionally, a display 86, such as an indicator light or a screen, may be provided on front control panel 82. Display 86 may be in communication with controller 84 and may display information in response to signals from controller 84.

As used herein, “processing device” or “controller” may refer to one or more microprocessors or semiconductor devices and is not restricted necessarily to a single element. The processing device can be programmed to operate dryer appliance 10. The processing device may include, or be associated with, one or more memory elements (e.g., non-transitory storage media). In some such embodiments, the memory elements include electrically erasable, programmable read only memory (EEPROM). Generally, the memory elements can store information accessible processing device, including instructions that can be executed by processing device. Optionally, the instructions can be software or any set of instructions and/or data that when executed by the processing device, cause the processing device to perform operations. For certain embodiments, the instructions include a software package configured to operate appliance 10 and execute certain cycles or operating modes.

In some embodiments, dryer appliance 10 also includes one or more sensors that may be used to facilitate improved operation of dryer appliance. For example, dryer appliance 10 may include one or more temperature sensors which are generally operable to measure internal temperatures in dryer appliance 10 and/or one or more airflow sensors which are generally operable to detect the velocity of air (e.g., as an air flow rate in meters per second, or as a volumetric velocity in cubic meters per second) as it flows through the appliance 10. In some embodiments, controller 84 is configured to vary operation profiles of dryer appliance 10 based on one or more temperatures detected by the temperature sensors or air flow measurements from the airflow sensors.

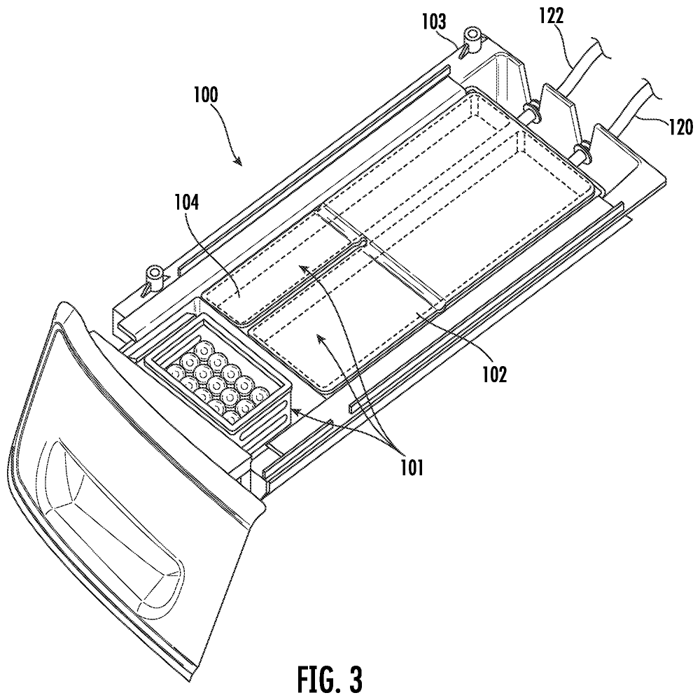

Referring now specifically to FIG. 3, a drawer 100 is provided according to one exemplary embodiment of the present disclosure. Drawer 100 may include one or more compartments 101. Compartments 101 may be used for storing treatment materials, including but not limited to sanitizing or scenting agents. Sanitizing agents are typically provided as a disinfectant fluid that may be poured into the one or more compartments 101. Disinfecting sanitizing agents typically are provided in concentrated form, although this is not necessary for the purposes of the present disclosure. Scenting agents may also be stored in one or more compartments 101. Commercially available scenting agents may take the form of a scented fluid solution or scented pellets. In the embodiment of FIG. 3, drawer 100 includes three compartments. First compartment 102 is a bulk storage tank for storing concentrated disinfecting fluids. Second compartment 104 is also a bulk storage tank for storing scented liquids used to scent articles of laundry during various drying cycles. In alternative embodiments, drawer 100 may include only a single compartment 101 for a single dose of disinfecting fluids. One of ordinary skill in the art will recognize that the number of compartments 101, what is stored in said compartments 101, and the size, shape, and geometric configuration of said compartments is not critical to the present disclosure and that various combinations are understood to fall within the scope of the present disclosure.

Drawer 100 may reside in a housing 103. Housing 103 may be fixed within cabinet 12 and include rails (not pictured) along which drawer 100 may slide. Housing 103 may further include an open side aligned with front panel 14, thereby allowing drawer 100 to slide through the open side of housing 103 and move between a closed position, wherein drawer 100 is contained within cabinet 12, and an open position, wherein drawer 100 extends from front panel 14 of cabinet 12. When in the open position, the one or more compartments 101 of drawer 100 become accessible from the exterior of dryer appliance 10. Movable covers or doors may, in some embodiments, enclose compartments 101 and, by opening the covers or doors, enable compartments 101 to be filled with the appropriate treatment materials.

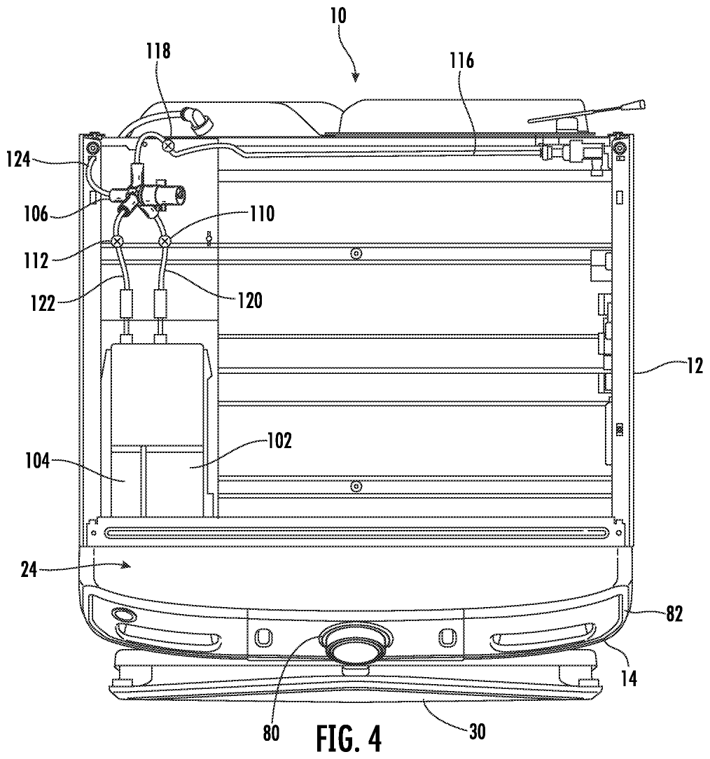

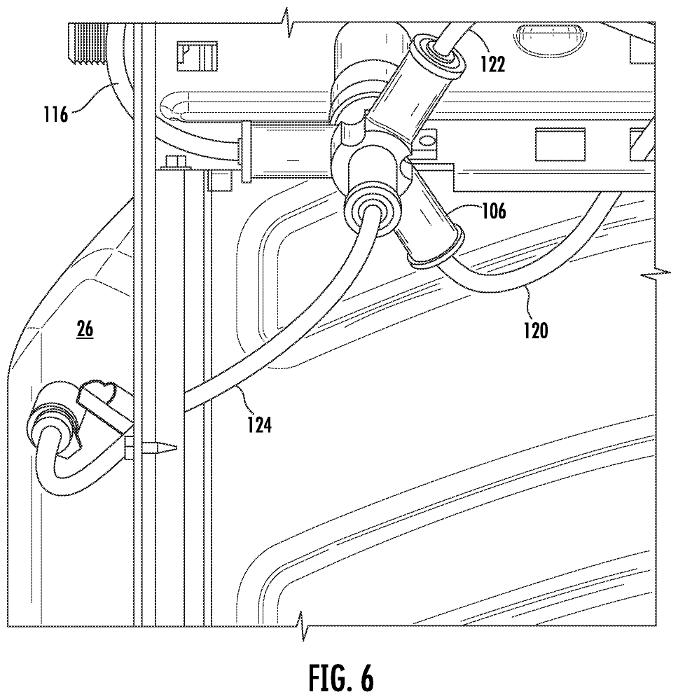

Each compartment 101 may have a separate inlet conduit attached for the transportation of the treatment materials therein. For example, in the embodiment of FIG. 4, first compartment 102 is connected to and in fluid communication with first inlet conduit 120. Similarly, second compartment 104 is connected to and in fluid communication with second inlet conduit 122. First inlet conduit 120 and second inlet conduit 122 are also connected to a pump 106 (FIGS. 4-6). As a result, pump 106 is also in fluid communication with first compartment 102 and second compartment 104. In certain embodiments, one or more valves may be interposed between compartments 101 and pump 106.

In embodiments employing valves, the number of valves will typically be equal to the number of compartments 101 in drawer 100, but this is not necessarily required. Indeed, some embodiments of the present disclosure may contain no valves whatsoever, but rather rely on the action of pump 106. Although the present disclosure describes embodiments involving a diaphragm pump, one of ordinary skill in the art will recognize that a wide variety of pumps could be used consistent with the disclosure herein, including but not limited to gear pumps, air pumps, peristaltic pumps, Venturi vacuums, and the like.

In the embodiments of FIGS. 4-6, a first valve 110 is interposed between first compartment 102 and pump 106. Additionally, second valve 112 is interposed between second compartment 104 and pump 106. First valve 110 and second valve 112 may be one-way electro-mechanical valves (for example, solenoid valves). In the embodiment of FIG. 4, first valve 110 and second valve 112 control the flow of treatment materials from first compartment 102 and second compartment 104, respectively, to pump 206.

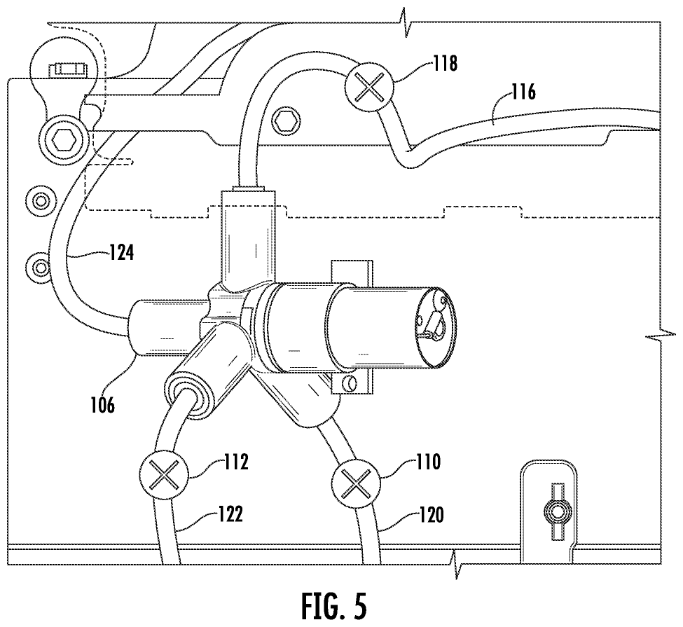

In certain embodiments, it may be desirable to dilute the treatment materials using water. In still other embodiments, pump 106 may be employed to circulate water alone. In the embodiment of FIG. 4, pump 106 may further be fed by water supply line 116, which provides water to pump 106 from a domestic water supply (e.g., city water or well). In alternative embodiments, the source of water may be an internal or external tank. Many conventional dryer appliances are already configured to accept a domestic water supply, which is typically used to supply steam for de-wrinkling and/or sanitization functions. As with the control of treatment materials from compartments 101, water supply line 116 may be interrupted by a water valve 118 (FIGS. 4-5). Water valve 118 controls the flow of water from the domestic water supply to pump 106. In embodiments that include a plurality of valves, one for each of the compartments 101, it will be recognized that the addition of the water valve 118 ensures that the quantity of valves is equal to or greater than the plurality of compartments 101 and each of the plurality of valves controls the flow of treatment materials from one of the plurality of compartments 101.

Still referring to the embodiments of FIGS. 4-6, an outlet conduit 124 is attached at one end to pump 106 and at the other end to a fluid distribution element 108. Outlet conduit 124 delivers treatment materials from pump 106 to fluid distribution element 108 such that fluid distribution element 108 is in fluid communication with pump 106. Fluid distribution element 108 may be a nozzle or atomizer mounted to, and extending through, drum 26. As such fluid distribution element 108 dispenses, in this embodiment, treatment materials and water into chamber 28 for treatment of articles of laundry therein (FIG. 7).

Although the embodiments of FIGS. 4-6 are provided with only a single pump, one of ordinary skill in the art will recognize that the present disclosure would equally apply if a plurality of pumps 106 were substituted for the single pump 106 of the FIGS. 4-6 embodiments. In particular, in some embodiments, a separate pump 106 may be used in association with each compartment 101 of drawer 100, in addition to another pump 106 used in association with water supply line 116, such that the quantity of the plurality of pumps 106 is equal to or greater than the plurality of compartments 101 and each of the plurality of pumps 106 is in fluid communication with the fluid distribution element 108 and one of the plurality of compartments 101. Even when a plurality of pumps is used, however, the number of pumps 106 need not equal the number of compartments 101 plus the water supply line 116. For example, a single pump 106 may be used in association with compartments 101 with a separate pump 106 for water supply line 116. Alternatively, a single pump 106 may be used in association with first compartment 102 and water supply line 116 with another pump 106 used in association with one or more additional compartments 101. The skilled artisan will recognize that the present disclosure is not limited to the particular embodiments described herein but extends to any various combination of pumps 106 in association with any number of compartments 101 and water supply line 116. It will further be recognized that the number of pumps 106 and the number of compartments 101 will also affect the number inlet conduits from the one or more compartment 101 to the one or more pumps 106. Likewise, the number of outlet conduits 124 will also be affected by the number of pumps, as will be clear to the skilled artisan.

Although dryer appliance 10 is described herein in the context of a front-loading dryer, this description is provided solely for illustrative purposes. According to the present disclosure, the described sanitization may be incorporated into a variety of dryer appliances. For instance, the present disclosure may be installed within a front-load dryer appliance, a top-load dryer appliance, or a combination washing machine and dryer. Advantageously, the features of the described sanitization system may significantly speed up the sanitization process (e.g., sanitization within 15 minutes as opposed to 90 minutes) with more efficient energy consumption than existing dryer sanitization cycles. However, additional uses will be apparent to one having ordinary skill in the art, such as the application of scents to laundered items, as discussed herein.

With the structure of various embodiments of dryer appliance 10 described above, embodiments describing the operation of dryer appliance 10 will now be addressed. Operation of laundry appliance 10 begins with the user selecting a cycle setting using selector input 80 of control panel 82. Dryer appliance 10 may include one or more cycle settings that seek to sanitize the articles of laundry. For example, dryer appliance 10 may include a cycle setting dedicated to performing sanitization. Additionally, or alternatively, dryer appliance 10 may include one or more cycle settings for which sanitization is only a portion of the intended functionality (e.g., a drying cycle followed by sanitization cycle). In still other embodiments, dryer appliance 10 may include one or more cycle settings that prompt the user whether a sanitization cycle is wanted at the end of another cycle.

Following selection of a cycle setting that includes sanitization, controller 84 initiates operation of the selected cycle. Controller 84, as previously discussed, controls operation and sequencing of the functionality of dryer appliance 10. Controller 84, for example, may be in communication with motor 27 for rotating drum 26, fan 32 for cycling air through chamber 28, heater 39 for heating such air, pump 106 for motivating treatment materials from drawer 100 to fluid distribution element 108, and water valve 118. Controller 84 may further be in communication with valves for controlling the flow of treatment materials from compartments 101 to pump 106, such as first valve 110 and second valve 112 in the embodiment of FIG. 4.

Upon selection of a sanitization cycle (or reaching the sanitization portion of a cycle if combined with other functionality), controller 84 is configured to control these elements of dryer appliance 10, fundamentally, to introduce the contents of compartments 101 of drawer 100 into chamber 28. The elements activated, and the sequencing of such activations, depends on a number of factors, such as the cycle setting and/or a fan setting.

For example, in some embodiments, the cycle setting selected by the user may call for sanitization of articles of laundry after a drying cycle. As the drying cycle draws to a close, fan 32 may be running to circulate heated air through chamber 28. Fan 32 may have at least two fan settings: on or off. In other embodiments, additional fan settings are possible, such as operation of fan 32 at different speeds. Upon transitioning to a sanitization cycle, it may be preferable to switch the fan setting of fan 32 to off to encourage that a maximum of amount of treatment fluids to be introduced remain within chamber 28 during the application process. In this embodiment, at the initiation of the sanitization cycle, controller 84 would deactivate fan 32. Only after deactivating fan 32 would controller 84 open one or more valves controlling the flow of treatment materials from compartments 101. Controller 84 may also open water valve 118 and activate pump 106 enabling the flow of water and treatment materials from compartments 101 through pump 106 and into chamber 28 through fluid distribution element 108.

Considering an alternative embodiment, such as the embodiment of FIG. 4, and selection of cycle setting requiring sanitization and scenting of articles of laundry, first compartment 102 may contain a sanitizing agent and second compartment 104 may contain a scenting agent. As before, operation of fan 32 in this embodiment may undesirably carry the treatment materials being introduced out of chamber 28 and through the exhaust system prior to reaching the articles of laundry intended to be treated. Thus, controller 84 would deactivate fan 32, if necessary, based on the current fan setting.

The controller 84 would then open first valve 110 and water valve 118, as well as activate pump 106, causing the sanitizing agent in first compartment 102 to be discharged into chamber 28 by fluid distribution element 108. In this way, it will be apparent that opening of first valve 110 is based on both the fan setting (e.g., whether fan 32 is on or off) and the cycle setting (e.g., whether the cycle calls for operation of fan 32, heater 39, and/or tumbling of drum 26, as further discussed herein).

After closing first valve 110, indicating an end to the sanitization phase, controller 84 would then open second valve 112, causing the scenting agent in second compartment 104 to be discharged into chamber 28 by fluid distribution element 108. In alternative embodiments, the sanitizing agent may not be concentrated and opening of water valve 118, or even the presence of water valve 118, may be unnecessary in conjunction with opening of first valve 110. The opening of water valve 118 could also be omitted in certain embodiments involving the opening of second valve 112.

In still other embodiments, other cycle settings may call for activation or deactivation of additional elements of dryer appliance 10 in various sequences. For example, a cycle setting may exist for sanitization of dryer appliance 10 itself, rather than articles of laundry therein. With this cycle setting, it would be preferable to use an “on” fan setting of fan 32, causing treatment materials introduced into chamber 28 to circulate out of chamber 28 and through the exhaust portions of dryer appliance 10. In such embodiments, controller 84 would activate fan 32 prior to opening first valve 110.

In further embodiments, a cycle setting may exist to sanitize articles of laundry at high or low temperatures, for example, due to the types of articles of laundry being sanitized and their susceptibility to damage as a result of heat. In such embodiments, controller 84 would activate or deactivate, respectively, heater 39 prior to opening of first valve 110 may be necessary. In still other embodiments, a cycle setting may call for sanitization in combination with prevention of tumbling of the articles of laundry in drum 26. Such an embodiment may be useful with delicate and/or hard articles of laundry, such as shoes. In such embodiments, controller 84 would deactivate motor 27 prior to opening first valve 110, thereby ensuring that drum 26 did not rotate during the treatment process. One of ordinary skill will recognize that the above exemplary embodiments could be used in combination or independently to arrive at different embodiments, all of which are within the intended scope of the present disclosure.

The above disclosure addresses operations performed in a particular order for purposes of illustration and discussion. Those of ordinary skill in the art, using the disclosures provided herein, will understand that the operations discussed herein can be adapted, rearranged, expanded, omitted, or modified in various ways without deviating from the scope of the present disclosure. Moreover, although aspects of the operation of dryer appliance 10 are explained using the embodiment of FIG. 4 as an example, it should be appreciated that these operations may be applied to the operation of any suitable dryer appliance.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A dryer appliance comprising:
a cabinet having a front side;
a rotatable drum within the cabinet, the rotatable drum defining a chamber for the receipt of articles for drying;
a drawer on the front side of the cabinet being slideable between a closed position and an open position, the drawer including a compartment for holding treatment materials, at least one of the treatment materials being a sanitizing agent;
a pump in fluid communication with the compartment of the drawer; and
a fluid distribution element for dispensing treatment materials into the chamber, wherein the fluid distribution element is in fluid communication with the pump.

2. The dryer appliance of claim 1, the dryer appliance further comprising a first valve interposed between the compartment and the pump for controlling the flow of treatment materials.

3. The dryer appliance of claim 2, the dryer appliance further comprising a water supply line for providing water from a domestic water supply to the pump.

4. The dryer appliance of claim 3, the dryer appliance further comprising a water valve in the water supply line for controlling the flow of water.

5. The dryer appliance of claim 4, the dryer appliance further comprising a fan for circulating air through the chamber, and the fan includes a fan setting of on or off.

6. The dryer appliance of claim 5, wherein opening of the first valve is based on the fan setting and a cycle setting.

7. The dryer appliance of claim 1, wherein drawer includes a plurality of compartments.

8. The dryer appliance of claim 7, the dryer appliance further comprising a plurality of pumps, wherein the quantity of the plurality of pumps is equal to or greater than the plurality of compartments and each of the plurality of pumps in fluid communication with the fluid distribution element and one of the plurality of compartments.

9. The dryer appliance of claim 8, the dryer appliance further comprising a plurality of valves, wherein the quantity of the plurality of valves is equal to or greater than the plurality of compartments and each of the plurality of valves controls the flow of treatment materials from one of the plurality of compartments.

10. The dryer appliance of claim 1, wherein the fluid distribution element is an atomizer.

11. A dryer appliance comprising:
a cabinet have a front side;
a rotatable drum within the cabinet, the rotatable drum defining a chamber for the receipt of articles for drying;
a fan for circulating air through the chamber;
a drawer on the front side of the cabinet being slideable between a closed position and an open position, the drawer including a first compartment and a second compartment for holding treatment materials;
one or more pumps in fluid communication with the first compartment and the second compartment of the drawer;
a fluid distribution element for dispensing treatment materials into the chamber, wherein the fluid distribution element is in fluid communication with the one or more pumps;
a first valve interposed between the first compartment and one of the one or more pumps for controlling the flow of treatment materials;
a second valve interposed between the second compartment and one of the one or more pumps for controlling the flow of treatment materials; and
a controller in communication with the fan, the one or more pumps, the first valve, and second valve, wherein, during a dryer cycle, the controller is configured to:
deactivate the fan;
activate the pump;
open the first valve after deactivating the fan;
close the first valve;
open the second valve after closing the first valve.

12. The dryer appliance of claim 11, wherein the first compartment is configured to hold sanitizing fluids and the second compartment is configured to hold a scented material.

13. The dryer appliance of claim 11, the dryer appliance further comprising a water supply line for providing water from a domestic water supply to the pump.

14. The dryer appliance of claim 13, the dryer appliance further comprising a water valve in the water supply line for controlling the flow of water.

15. The dryer appliance of claim 14, the dryer appliance further comprising a first inlet conduit for delivering treatment material from the first compartment to the one or more pumps and a second inlet conduit for delivering treatment materials from the second compartment to the one or more pumps.

16. The dryer appliance of claim 15, wherein the one or more pumps includes separate pumps attached to the first inlet conduit, the second inlet conduit, and the water supply line.

17. The dryer appliance of claim 11, the dryer appliance further comprising an outlet conduit for delivering treatment materials from the one or more pumps to the fluid distribution element.

18. The dryer appliance of claim 11, wherein the fluid distribution element is a nozzle.

19. The dryer appliance of claim 18, wherein the nozzle is an atomizer.

20. The dryer appliance of claim 11, the controller further configured to open the water valve when the first valve is open.
